(19) United States
(12) Patent Application Publication
(10) Pub. No.: US 2018/0280632 A1
(43) Pub. Date: Oct. 4, 2018

(54) SYRINGE WARMING ASSEMBLY AND APPARATUS FOR SAME

(71) Applicant: JAMES G. GETSAY, HARMONY, PA (US)

(72) Inventor: JAMES G. GETSAY, HARMONY, PA (US)

(21) Appl. No.: 15/937,072

(22) Filed: Mar. 27, 2018

Related U.S. Application Data
(60) Provisional application No. 62/477,633, filed on Mar. 28, 2017.

(51) Int. Cl. A61M 5/44

(52) U.S. Cl. CPC A61M 5/44; A61M 5/3129

(57) ABSTRACT

An apparatus is for warming a syringe. The apparatus includes a corrugated tube member, a number of heating elements, and an insulating material. The corrugated tube member has a plurality of alternating ridge portions, a plurality of alternating furrow portions each located between a corresponding pair of the ridge portions, and a central bore structured to receive the syringe. The number of heating elements are provided within one or more of the furrow portions. The insulating material is provided around the corrugated tube member and encapsulates the number of heating elements within the furrow portions.

### CROSS-REFERENCE TO RELATED APPLICATION

This application claims priority from and claims the benefit of U.S. Provisional Patent Application Ser. No. 62/477,633, filed Mar. 28, 2017, and entitled “SYRINGE WARMER”.

### BACKGROUND OF THE INVENTION

### 1. Field of the Invention

The disclosed concept relates to apparatus for warming syringes and their contents prior to delivering the contents to a patient, and, in particular, to such an apparatus that includes a corrugated tube member with integrated heating elements. The disclosed concept also relates to syringe warming assemblies including such apparatus.

### 2. Description of the Related Art

Interstitial cystitis/bladder pain syndrome (IC/BPS) is a chronic pain condition that affects the bladder. Symptoms of IC/BPS include feeling the need to urinate right away, needing to urinate often, and pain with sex. The cause of IC/BPS is unknown.

When a person experiences an episode of IC/BPS, their doctor usually prescribes an antibiotic to rule out a urinary tract infection (UTI). Once a UTI has been ruled out, a transurethral irrigation, commonly referred to as a “wash”, is generally administered. The “wash” can range from FDA approved drugs (e.g., a dimethyl sulfoxide (DMSO)/heparin blend) to what urologists refer to as a “smoothie”, which is typically a mixture of their choice, usually involving anti-inflammatory/anti-coagulant agents.

The patient population for IC/BPS ranges into the millions in the U.S. alone, with many patients being trained to “self-irrigate” at home. Patients that self-irrigate may receive in the mail a pre-loaded syringe from their physician. At best, the temperature of the syringe is room temperature. Studies have shown, however, that certain agents, heparin in particular, are most effective when delivered normothermically (i.e., at normal body temperature). While there are expensive syringe warmers/heaters on the market for hospital/lab use, none are designed for in-home use.

### SUMMARY

As one aspect of the disclosed concept, an apparatus is provided for warming a syringe. The apparatus includes a corrugated tube member, a number of heating elements, and an insulating material. The corrugated tube member has a plurality of alternating ridge portions, a plurality of alternating furrow portions each located between a corresponding pair of the ridge portions, and a central bore structured to receive the syringe. The number of heating elements are provided within one or more of the furrow portions. The insulating material is provided around the corrugated tube member and encapsulates the number of heating elements within the furrow portions.

As another aspect of the disclosed concept, a syringe warming assembly is provided. The syringe warming assembly includes a syringe and the aforementioned apparatus.

### BRIEF DESCRIPTION OF THE DRAWINGS

Exemplary embodiments of the present disclosed concept are shown in the enclosed drawings as follows:

FIG. 1 is an isometric view of a syringe warming assembly and apparatus for the same, shown connected to a power source, in accordance with one non-limiting embodiment of the disclosed concept;

FIG. 2 is a partially exploded isometric view of the assembly and apparatus for the same of FIG. 1, shown without the syringe, centering member, and shell;

FIG. 3 is an isometric view of a corrugated tube member for the assembly and apparatus for the same of FIG. 1;

FIG. 4 is a section view of the assembly and apparatus for the same of FIG. 1;

FIG. 5 is an exploded section view of the assembly and apparatus for the same of FIG. 1, shown with an alternative stabilizing member;

FIGS. 6 and 7 are top and front section views, respectively, of the centering member for the apparatus of FIG. 1;

FIG. 8 is an exploded section view of the shell for the assembly and apparatus for the same of FIG. 1; and

FIG. 9 is an assembled section view of a portion of the shell of FIG. 8.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As employed herein, the statement that two or more parts or components “engage” one another shall mean that the parts exert a force against one another either directly or through one or more intermediate parts or components.

As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the subject invention. It will be evident, however, that the disclosed concept can be practiced without these specific details without departing from the spirit and scope of this innovation.

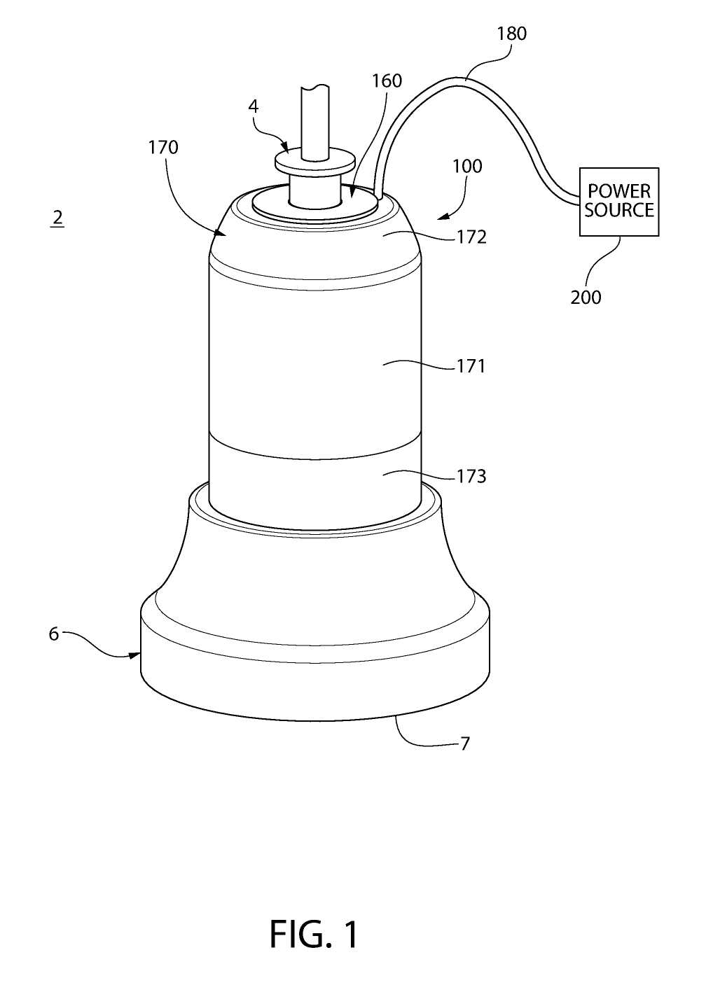

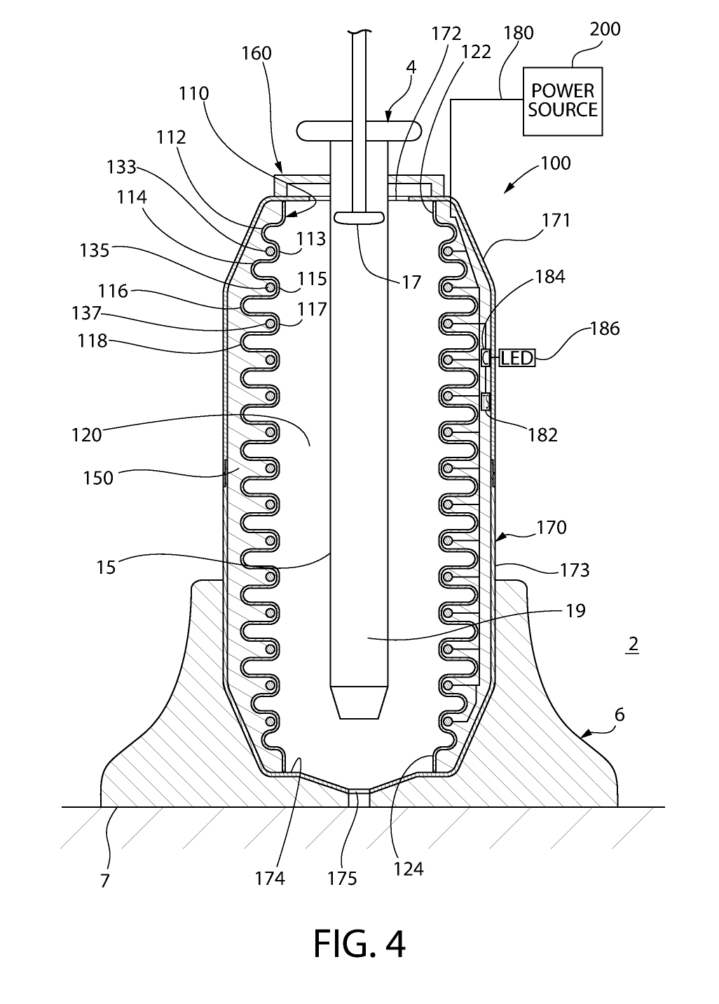

FIG. 1 shows an isometric view of a portion of a syringe warming assembly 2, in accordance with one non-limiting embodiment of the disclosed concept. The syringe warming assembly 2 includes a syringe 4, an example stabilizing member 6, and a novel syringe warming apparatus 100. As will be discussed in greater detail below, among other advantages, the apparatus 100 is configured to cause the syringe 4 (e.g., and the contents within the syringe 4) to be heated to a specific, predetermined temperature (e.g., without limitation, human normothermic temperature). The example syringe 4 includes a barrel 15 (shown in FIG. 4), and a plunger 17 (shown in FIG. 4) provided in the barrel 15 to selectively move and cause contents (see, for example, location 19 in FIG. 4) in the barrel 15 to be dispensed.

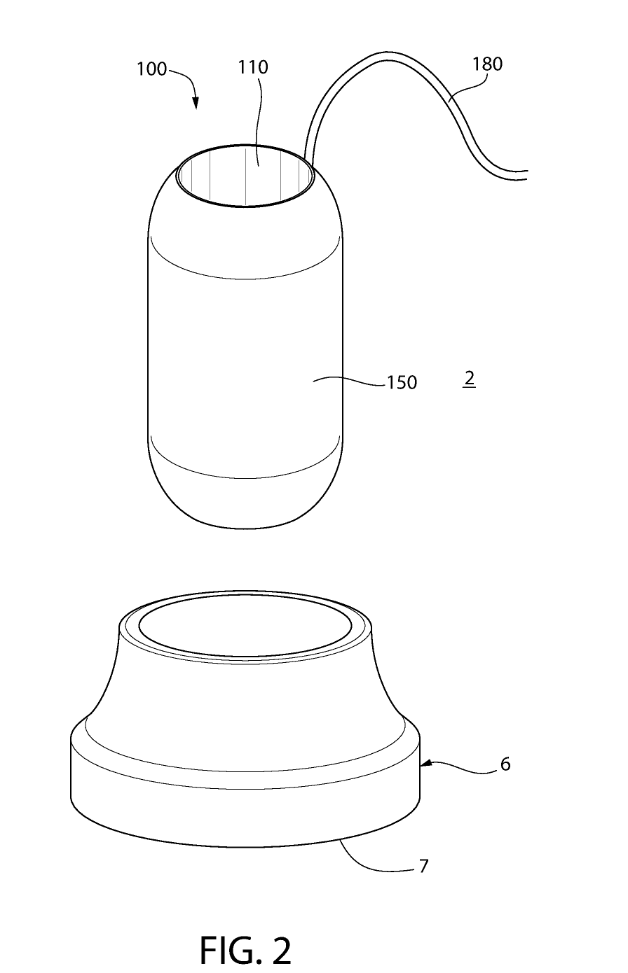

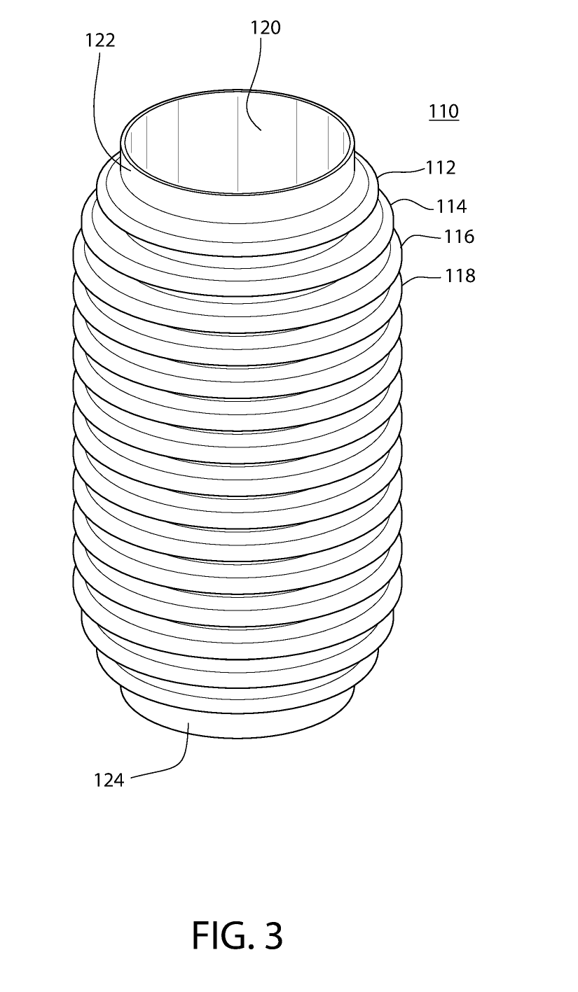

FIG. 2 shows an exploded view of a portion of the syringe warming assembly 2 of FIG. 1, shown without the syringe 4, and without a centering member 160 and a shell 170 of the apparatus 100. As shown, the apparatus 100 preferably includes a corrugated tube member 110 (partially shown in FIG. 2, but see also, for example, FIG. 3) and a thermally insulating material 150 provided around the corrugated tube member 110. In one example embodiment, the insulating material 150 is a silicone rubber material or another elastomeric insulating material. The example corrugated tube member 110 may be made of a thermally conductive material, such as, without limitation, a thermally conductive metal like stainless steel or aluminum.

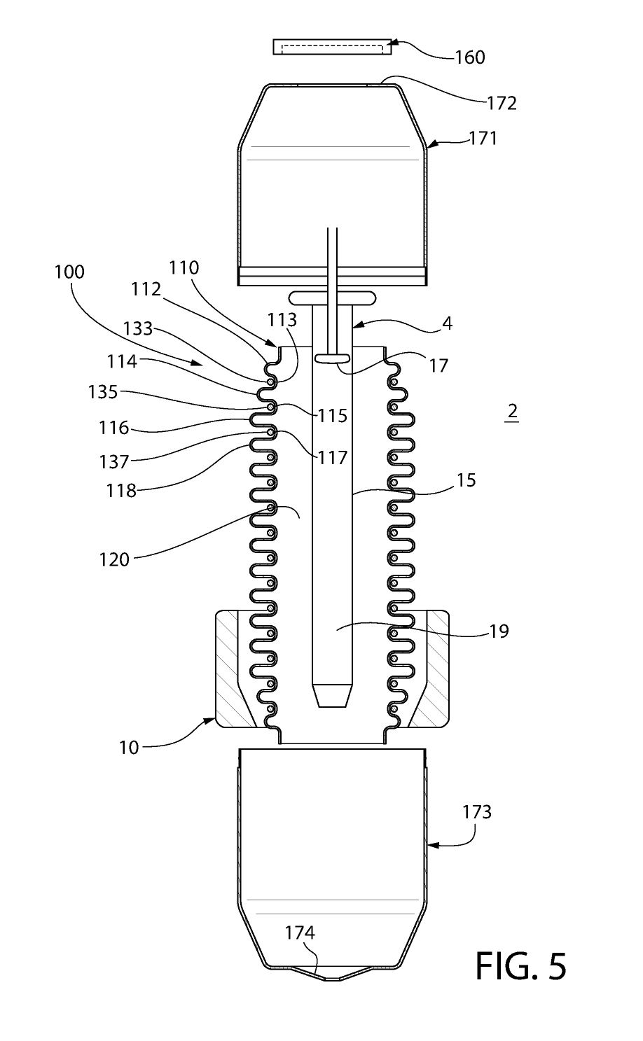

As shown in FIG. 3, the corrugated tube member 110 has a plurality of alternating ridge portions (four example ridge portions 112,114,116,118 are indicated in FIG. 3). Referring to FIG. 4, which is a section view of the assembly 2 of FIG. 1, the corrugated tube member 110 further has a plurality of alternating furrow (e.g., recessed and/or grooved) portions (three example furrow portions 113,115,117 are indicated in FIG. 4) each located between a corresponding pair of the ridge portions 112,114,116,118, and a central bore 120 that is structured to receive the syringe 4. See also, for example, FIG. 5, which shows the assembly 2 of FIG. 1, but with an alternative stabilizing member 10, and with certain components (e.g., without limitation, insulating material 150 and certain electrical components) removed for ease of illustration. While the corrugated tube member 110 is provided herein with the furrow portions 113,115,117 being substantially curved, it is within the scope of the disclosed concept for a suitable alternative corrugated tube member (not shown) to have substantially rectangular-shaped furrow portions.

Continuing to refer to FIG. 4, the apparatus 100 further includes a number of heating elements. In one example embodiment, the number of heating elements is a plurality of wires (three example wires 133,135,137 of the plurality of wires are indicated in FIG. 4) each provided within a corresponding one of the furrow portions 113,115,117. It is also within the scope of the disclosed concept for a suitable alternative syringe warming apparatus (not shown) to include a single heating element provided within a plurality of the furrow portions. As shown in FIG. 4, the insulating material 150 is provided around the corrugated tube member 110 and encapsulates the wires 133,135,137 within the furrow portions 113,115,117. As such, the insulating material 150 covers (e.g., surrounds on substantially all sides) each of the ridge portions 112,114,116,118 and each of the furrow portions 113,115,117. Accordingly, as shown, each of the plurality of wires 133,135,137 is substantially surrounded on all sides by the insulating material 150 and the corrugated tube member 110. As each of the wires 133,135,137 is preferably located in a respective one of the furrow portions 113,115,117, as will be discussed below, the wires 133,135,137 are thus configured to pass heat into the central bore 120 of the corrugated tube member 110 in order to heat the syringe 4.

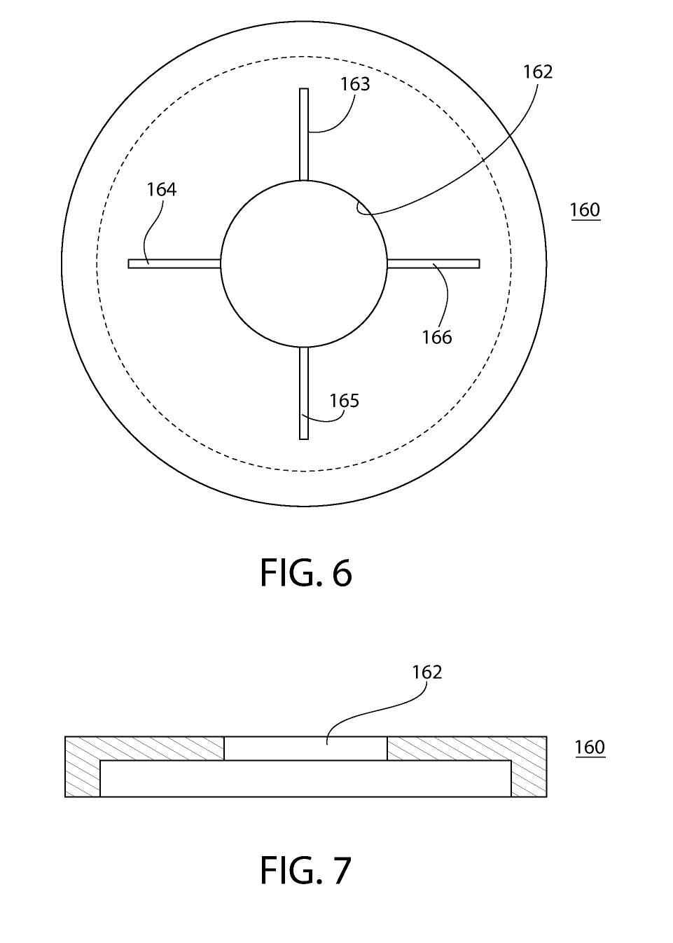

The apparatus 100 also further includes a centering member 160. The corrugated tube member 110 further has a first end 122 and a second end 124 located opposite and distal the first end 122. As shown, the centering member 160 is coupled indirectly to the first end 122 of the corrugated tube member 110. Referring to FIGS. 6 and 7, the centering member 160 has a central edge portion 162 defining a thru hole in the centering member 160, and a number of slits 163,164,165,166 extending radially outwardly from the edge portion 162. It will be appreciated with reference to FIG. 4 that the thru hole defined by edge portion 162 advantageously receives and centers the syringe 4 within the central bore 120 of the corrugated tube member 110, and the slits 163,164,165,166 allow for some flexibility as the syringe 4 is inserted through the edge portion 162. As a result, when the syringe 4 is located internal with respect to the apparatus 100, as depicted in FIG. 4, the syringe 4 does not engage the corrugated tube member 110. Accordingly, the integrity of the body of the syringe 4 will generally not be compromised by the apparatus 100, and the apparatus 100 will advantageously be able to heat the syringe 4 via indirect exposure to the corrugated tube member 110 and the wires 133,135,137.

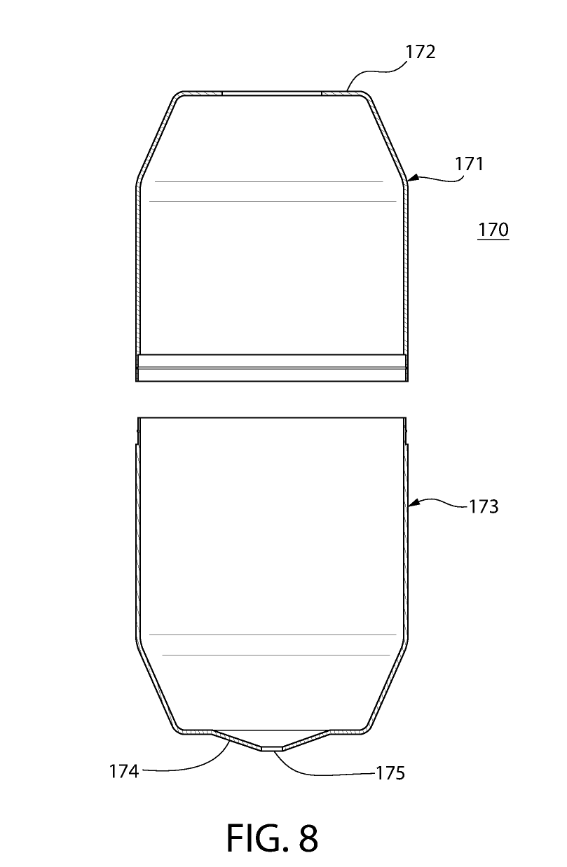

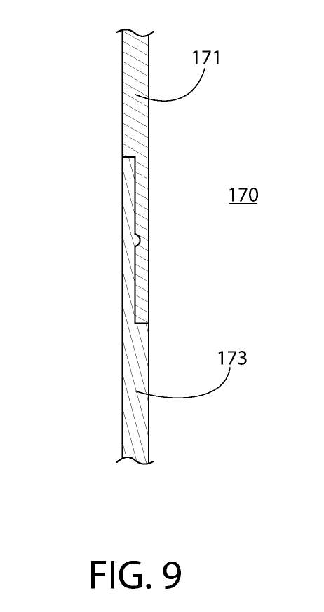

The apparatus 100 also includes a shell 170 coupled to and substantially enclosing the insulating material 150. It will be appreciated with reference to FIGS. 4 and 5 that the centering member 160 is preferably directly coupled to the shell 170. In one example embodiment, the centering membrane 160 is coupled to the shell 170 by a snap-fit mechanism. FIG. 8 shows an exploded section view of the shell 170, and FIG. 9 shows an assembled section view of a portion of the shell 170 of FIG. 8. As shown in FIG. 8, the shell 170 has a first member 171 and a second member 173. The first member 171 has a first end 172, and the second member 173 has a second end 174 located opposite and distal the first end 172. The first end 172 is configured to be located proximate the first end 122 (FIG. 4) of the corrugated tube member 110, and the second end 174 is located proximate the second end 124 (FIG. 4) of the corrugated tube member 110. Additionally, as shown in FIG. 9, in one example embodiment the first member 171 is directly coupled to the second member 173 by a snap-fit mechanism. It will, however, be appreciated that suitable alternative coupling mechanisms may be employed, without departing from the scope of the disclosed concept.

It will also be appreciated with reference to FIG. 4 that the corrugated tube member 110 and the insulating material 150 are each located internal with respect to the first and second members 171,173. As such, when the apparatus 100 is assembled, the shell 170 provides a beneficial mechanism to protect the internal components (e.g., without limitation, insulating material 150, wires 133,135,137, corrugated tube member 110, and electrical components) from contact with external forces that might otherwise compromise their integrity. For example, the shell 170 may be made of a rigid thermoplastic material, a metallic material, or any other suitable material. Accordingly, as the insulating material 150 may be relatively soft, including with the apparatus 100 the relatively rigid shell 170 protects the insulating material 150 from forces that would otherwise cause the material to wear away. As a result, the life of the apparatus 100 is advantageously able to be prolonged with the shell 170.

Additionally, the shell 170 also has a mechanism to allow contents of the syringe 4, which might otherwise become entrapped in the apparatus 100, to escape. Specifically, as shown in FIG. 4, the second end 174 of the shell 170, which substantially extends across the central bore 120 proximate the second end 124 of the corrugated tube member 110, has a central thru hole. Furthermore, in one example embodiment the second end 174 is funnel-shaped and has an apex portion (e.g., without limitation, edge portion 175) located opposite and distal the first end 172 of the shell 170. As shown, the central through hole is defined by, or is located at, edge portion 175.

As shown in FIG. 4, the stabilizing member 6 has a planar portion 7 (e.g., a generally flat base). Furthermore, the second end 174 of the shell 170 is located generally internal with respect to the stabilizing member 6 in order to maintain the syringe 4 in a position perpendicular to the planar portion 7, which is configured to rest on and be parallel with the ground. As such, the stabilizing member 6, which due to its geometry (e.g., relatively narrow top and relatively wide base), is advantageously able to maintain the apparatus 100 in a vertical position. This is beneficial when, during warming of the syringe 4, it is best for the syringe to not directly engage the corrugated tube member 110.

FIG. 5 shows a section view of a portion of another stabilizing member 10, structured to perform the same function as the stabilizing member 6. However the stabilizing member 10 functions as, for example and without limitation, a pole or wall mount clamp that wraps around the shell 170 (not shown in FIG. 5, but see FIG. 4).

The functionality of the apparatus 100 will now be discussed in greater detail in association with FIG. 4. As mentioned above, the apparatus 100 is configured to cause the syringe 4 (e.g., and the contents 19 within the syringe 4) to be heated to a specific, predetermined temperature (e.g., without limitation, human normothermic temperature). In order to perform the function of heating the syringe 4, the apparatus 100 further includes a connector 180 (shown in simplified form in FIG. 4) electrically connected to the wires 133,135,137. The connector 180 may be, for example and without limitation, a USB chord. As shown, the connector 180 is also structured to be mechanically coupled and electrically connected to a power source 200 (shown in simplified form in FIG. 4). The power source 200 may be, for example and without limitation, an AC wall outlet. The apparatus 100 further includes a temperature sensor 182 (shown in simplified form) such as, without limitation, a thermistor. The apparatus 100 also further includes an integral controller 184 (shown in simplified form) electrically connected to the temperature sensor 182, and an indicating device (e.g., without limitation, light emitting diode (LED) 186, shown in simplified form) electrically connected to the controller 184. The controller 184 may be, without limitation, a microcontroller, a microprocessor, an application specific integrated circuit (ASIC), or any other suitable processing device. The temperature sensor 182 and the controller 184 are preferably embedded within or on the insulating material 150.

Accordingly, it will be appreciated that the controller 184 is structured and configured (i.e., programmed) to control operation of the wires 133,135,137 in order to cause the apparatus 100 to be heated to a specific, predetermined temperature. Specifically, as shown, the temperature sensor 182 and the controller 184 are each electrically connected to the wires 133,135,137 and to each other. Thus, when the connector 180 is connected to the power source 200, and current flows through, and raises the temperature of, the wires 133,135,137, the temperature sensor 182 is advantageously able to sense the temperature of the wires 133,135,137. Because the controller 184 controls the amount of current provided to the wires 133,135,137, and the temperature of the wires 133,135,137 is directly related to the temperature of the contents 19 within the barrel 15 of the syringe 4, the controller 184 can advantageously control the temperature of the contents 19 within the barrel 15.

In addition, in the embodiment provided for in FIG. 4, the controller 184 is further structured and configured (e.g., programmed) to control the LED 186 to provide a perceptible indicator (e.g., light given off by the LED 186) when the specific, predetermined temperature has been reached. Specifically, as shown, the LED 186 is electrically connected to the controller 184, and is positioned on an exterior of the apparatus 100. As such, a signal from the controller 184 to the LED 186 indicating that a predetermined temperature has been reached would cause the LED 186 to emit light, thus indicating to a user that the syringe 4 has properly been heated and is ready for use. While the disclosed concept has been described in association with the LED 186, it is within the scope of the disclosed concept to provide for a suitable alternative indication device (e.g., without limitation, an audio device that emits sound to provide an indication to a user that a predetermined temperature has been reached, or an LCD that generates and displays a message to a user indicating that a predetermined temperature has been reached).

In operation, a patient either receives the syringe 4 prefilled with the contents 19 (e.g., from a physician), or fills in the empty syringe 4 with the contents 19 provided by position. The patient then inserts the filled syringe 4 into the apparatus 100 and initiates heating by way of a button or the like coupled to the controller 184. The patient then waits while the wires 133,135,137 are powered and heat the apparatus 100 as described herein. When the indicating device (e.g., the LED 186) indicates that the specific, predetermined temperature has been reached as described herein, the patient removes the syringe 4 from the apparatus 100 and shortly thereafter begins self irrigation.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. An apparatus for warming a syringe, the apparatus comprising:
a corrugated tube member having a plurality of alternating ridge portions, a plurality of alternating furrow portions each disposed between a corresponding pair of the ridge portions, and a central bore structured to receive the syringe;
a number of heating elements provided within one or more of the furrow portions; and
an insulating material provided around the corrugated tube member and encapsulating the number of heating elements within the one or more of the furrow portions.

2. The apparatus according to claim 1, wherein the corrugated tube member has a first end and a second end disposed opposite and distal the first end; wherein the apparatus further comprises a centering member coupled to the first end; and wherein the centering member has a thru hole in order to receive and center the syringe within the central bore of the corrugated tube member.

3. The apparatus according to claim 2, wherein the apparatus further has a shell coupled to and substantially enclosing the insulating material; and wherein the centering member is coupled to the shell by a snap-fit mechanism.

4. The apparatus according to claim 3, wherein the shell has a first end and a second end disposed opposite and distal the first end of the shell; wherein the first end of the shell is disposed proximate the first end of the corrugated tube member; wherein the second end of the shell substantially extends across the central bore proximate the second end of the corrugated tube member; and wherein the second end of the shell has a central thru hole.

5. The apparatus according to claim 4, wherein the second end of the shell is funnel-shaped and has an apex portion disposed opposite and distal the first end of the shell; and wherein the central thru hole is disposed at the apex portion.

6. The apparatus according to claim 3, wherein the shell comprises a first member and a second member coupled to the first member by a snap-fit mechanism; and wherein the corrugated tube member is disposed internal with respect to each of the first member and the second member.

7. The apparatus according to claim 1, wherein the number of heating elements are a plurality of heating elements each provided within a respective one of the furrow portions.

8. The apparatus according to claim 7, wherein the plurality of heating elements are a plurality of wires.

9. The apparatus according to claim 1, wherein the number of heating elements is a single heating element provided within a plurality of the furrow portions.

10. The apparatus according to claim 1, wherein the insulating material covers each of the ridge portions and each of the furrow portions.

11. The apparatus according to claim 1, wherein the insulating material is silicone rubber.

12. The apparatus according to claim 1, further comprising a connector electrically connected to the number of heating elements; and wherein the connector is structured to be mechanically coupled and electrically connected to a power source.

13. The apparatus according to claim 12, further comprising a controller and a temperature sensor electrically connected to the controller; wherein the controller is structured and configured to control operation of the heating elements in order to cause the apparatus to be heated to a specific, predetermined temperature.

14. The apparatus according to claim 13, wherein the controller and the temperature sensor are embedded within the insulating material.

15. The apparatus according to claim 13, wherein the specific, predetermined temperature is human normothermic temperature.

16. The apparatus according to claim 13, further comprising an indicating device electrically connected to the controller; wherein the controller is further structured and configured to control the indicating device to provide a perceptible indicator when the specific, predetermined temperature has been reached.

17. The apparatus according to claim 16, wherein the indicating device is an LED.

18. A syringe warming assembly comprising:
a syringe; and
an apparatus for warming the syringe, the apparatus comprising:
a corrugated tube member having a plurality of alternating ridge portions, a plurality of alternating furrow portions each disposed between a corresponding pair of the ridge portions, and a central bore structured to receive the syringe,
a number of heating elements provided within one or more of the furrow portions, and
an insulating material provided around the corrugated tube member and encapsulating the number of heating elements within the one or more of the furrow portions.

19. The syringe warming assembly according to claim 18, wherein the apparatus further has a shell coupled to and substantially enclosing the insulating material; wherein the shell has a first end and a second end disposed opposite and distal the first end; wherein the corrugated tube member extends from proximate the first end to proximate the second end; wherein the syringe warming assembly further comprises a stabilizing member; wherein the stabilizing member has a planar portion; and wherein the second end of the shell is disposed generally internal with respect to the stabilizing member in order to maintain the syringe in a position perpendicular to the planar portion.

20. The syringe warming assembly according to claim 18, wherein the syringe does not engage the corrugated tube member.
